(19) United States
(12) Patent Application Publication
(10) Pub. No.: US 2007/0162041 A1
(43) Pub. Date: Jul. 12, 2007

(54) Instrument and system for preparing the disc space between two vertebral bodies

(75) Inventors: Bruce Robie, Glen Rock, NJ (US); Felix Blouin, Sterling Forest, NY (US); Rafael Zubok, Midland Park, NJ (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER TRABECULAR METAL TECHNOLOGY, INC., Allendale, NJ (US)

(21) Appl. No.: 11/656,765

(22) Filed: Jan. 23, 2007

Related U.S. Application Data
(63) Continuation of application No. 10/015,365, filed on Dec. 13, 2001, now abandoned.
(60) Provisional application No. 60/317,405, filed on Sep. 4, 2001.

(51) Int. Cl. A61B 17/58

(52) U.S. Cl. 606/90

(57) ABSTRACT

An instrument for distracting the disc space between adjacent vertebrae and simultaneously preparing endplates of the vertebrae includes a body having opposing upper and lower surfaces separated by curved side surfaces that extend between a posterior end of the body and an anterior end of the body. A first plurality of teeth extends across the upper surface of the body, and a second plurality of teeth extends across the lower surface of the body. The first and second plurality of teeth may constructed as ratcheting teeth that angle back toward the anterior end of the body. Also a system for distracting the disc space between adjacent vertebrae and simultaneously preparing endplates of the vertebrae, includes at least two differently dimensioned instruments as set forth above.

This application is a continuation of U.S. patent application Ser. No. 10/015,365, filed Dec. 13, 2001. This application also claims the benefit of U.S. Provisional Application Ser. No. 60/317,405 filed Sep. 4, 2001.

### FIELD OF THE INVENTION

The present invention relates to instruments and instrument systems for preparing the two endplates of a disc space for either insertion of a material and/or device intended to prevent motion between the two adjacent vertebrae (commonly referred to as fusion) or a device intended to permit motion between the two adjacent vertebral bodies (sometimes referred to as an artificial disc or artificial motion segment).

### BACKGROUND OF THE INVENTION

A large segment of the population have chronic back problems which cause pain and disability. The cause of back pain is often the result of diseased disc material between opposing vertebrae. The diseased disc material usually causes pain because the disc material inadequately supports the opposing vertebrae, thereby allowing the space between the vertebrae to collapse and irritate or damage a spinal nerves.

Surgical techniques have been developed to eliminate the persistent pain resulting from diseased disc material. In such techniques, the diseased disc material is removed and the joint between opposing vertebral bodies is fused. Fusion specifically involves removing the diseased disc, preparing a bore for receiving an implant, and inserting the implant between the opposing vertebral bodies.

Spinal fusion implants and the surgical instruments used for implanting these fusion implants are well known in the art. The surgical instruments required for preparing the disc space typically include one or more distractors, drills, reamers, and other instrument. The need for so many different instruments increases the complexity and cost of the surgery. Moreover the use of drills and reamers creates a risk of pushing tissue towards the neural structures.

Accordingly, an instrument which avoids the above problems is needed.

### SUMMARY OF THE INVENTION

An instrument is disclosed herein for distracting a disc space between adjacent vertebrae and simultaneously preparing endplates of the vertebrae. The instrument comprises a body having opposing upper and lower surfaces separated by curved side surfaces that extend between a posterior end of the body and an anterior end of the body. A first plurality of teeth extends across the upper surface of the body, and a second plurality of teeth extends across the lower surface of the body. The first and second plurality of teeth may be constructed as ratcheting teeth that angle back toward the anterior end of the body.

Also disclosed herein is a system for distracting a disc space between adjacent vertebrae and simultaneously preparing endplates of the vertebrae. The system comprises at least two differently dimensioned instruments as set forth above.

### BRIEF DESCRIPTION OF THE DRAWINGS

The advantages, nature, and various additional features of the invention will appear more fully upon consideration of the illustrative embodiments now to be described in detail in connection with accompanying drawings where like numerals are used to identify like elements and wherein:

FIG. 1 is a plan view of an exemplary embodiment of a combination broach and distractor instrument made according to the principles of the present invention;

FIG. 2 is a plan view of an exemplary embodiment of a broach inserter of the instrument of FIG. 1;

FIG. 3A is a plan view of an exemplary embodiment of a broach of the instrument of FIG. 1;

FIG. 3B is a posterior end view of the broach of FIG. 3A;

FIG. 3C is an anterior end view of the broach of FIG. 3A;

FIG. 4 is a side elevational view of the broach of FIG. 3A;

FIG. 5 is an enlarged side elevational view of an exemplary broach in a 7 or 8 mm size showing the two anterior-most teeth (of the upper or lower body surface); and

FIG. 6 is an enlarged side elevational view of an exemplary broach in a 9 to 18 mm size showing the two anterior-most teeth (of the upper or lower body surface).

It should be understood that the drawings are for purposes of illustrating the concepts of the invention and are not necessarily to scale.

### DETAILED DESCRIPTION OF THE INVENTION

FIG. 1 shows an exemplary embodiment of a combination broach and distractor instrument 10 made according to the principles of the present invention. The instrument 10 generally comprises a broach 12 and a broach inserter 14, both of which are typically made from stainless steel. One of ordinary skill in the art will of course appreciated that the instrument 10 may also be made from other suitable materials.

As will soon become apparent, the instrument 10 of the present invention is configured to prepare the disc space for a like shaped implant/bone graft i.e., a material and/or device intended to prevent motion between the two adjacent vertebrae (commonly referred to as fusion) or a device intended to permit motion between the two adjacent vertebral bodies (sometimes referred to as an artificial disc or artificial motion segment). The instrument 10 prepares the endplates of both vertebrae simultaneously so as to minimize the labor of surgery, and distracts the disc space as broaches 12 of increasing size are inserted into the disc space.

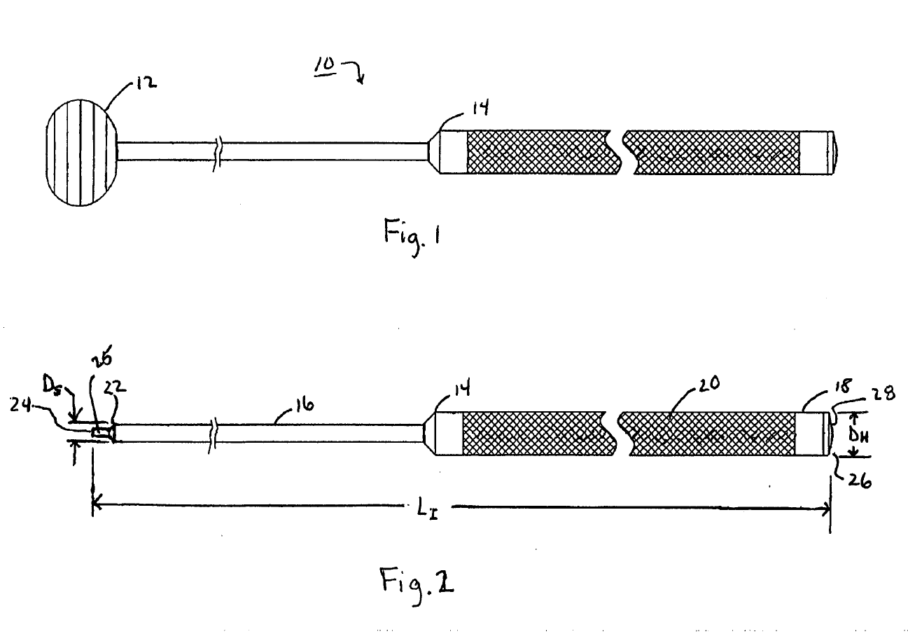

As shown in FIG. 2, the broach inserter 14 typically includes an elongated driver shaft 16 and a handle 18 having a diameter DH which may be greater than the diameter Ds of the shaft 16. The shaft 16 may be provided at a distal end 22 thereof with a fastening element 24 for removably coupling the broach 12 to the inserter 14. By way of example but not limitation, the fastening element 24 may comprise a threaded stud 25, extending axially therefrom. The handle 18 may be provided with a knurled surface 20 or other means for facilitating secure manual gripping of the handle 18. A proximal end 26 of the handle 18 is typically provided with a convex surface 28 against which a mallet or other like device may be used to tap the handle 18. In one exemplary embodiment, the inserter 14 may have a length LI of about 12.2 inches. In another exemplary embodiment, the inserter 14 may have a length LI of about 18.6 inches. One of ordinary skill in the art will of course appreciate that the inserter 14 may be made in other lengths.

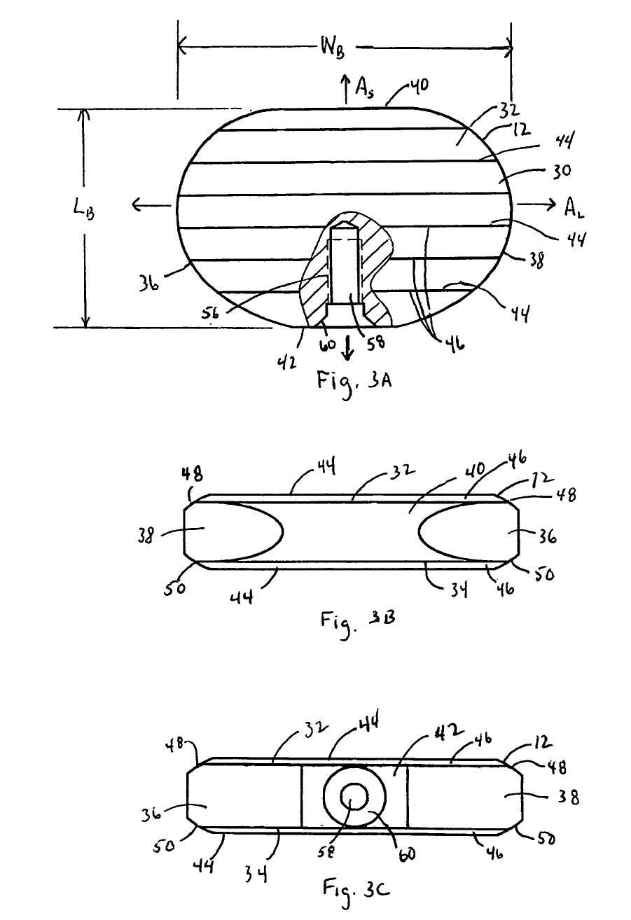

As shown in FIG. 3A, the broach 12, as viewed in plan, generally has a asymmetrically formed oval body 30 defining a short axis As that extends in an anterior-posterior direction, and a long axis AL that extends perpendicular to the short axis As. In one exemplary embodiment, the body 30 may have a length LB, as measured in the direction of the short axis As, of about 0.86 inches and a width WB, as measured in the direction of the long axis AL, of about 1.274 inches. In another exemplary embodiment, the body 30 may have a length LB of about 1.08 inches and a width WB of about 1.612 inches. One of ordinary skill in the art will of course appreciate that the body 30 may be made in other lengths LB and widths WB to match the device to be implanted.

The body 30 includes opposing upper and lower surfaces 32, 34 that are separated by curved side surfaces 36, 38 which extend axially between a truncated posterior end 40 of the body 30 as shown in FIG. 3B and a truncated anterior end 42 of the body 30 as shown in FIG. 3C. The curved side surfaces 36, 38 are shaped to mimic the overall U-shape of the vertebral bodies. The upper and lower surfaces 32, 34 of the body 30 each define one or more elements 44 that are configured to 1) distract the disc space between two vertebral bodies without cutting or shaving bone tissue when the broach is inserted therein, and 2) cut or shave soft tissue or bone when the broach 12 is withdrawn from the disc space, thereby minimizing the risk of pushing tissue towards neural structures.

The element or elements 44 are preferably configured as a plurality of ratcheting teeth 46. The teeth 46 typically extend across the upper and lower surfaces 32, 34 of the body 30, in a direction which is generally perpendicular to the short axis As of the body 30 and inserter 14. The teeth 46 preferably extend across the body 30 in a continuous manner. When viewed from either the posterior or anterior end 40, 42, the side surfaces 36, 38 appear substantially flat with beveled or curved upper and lower edges 48, 50 that merge into the ends of the teeth 46 defined by the upper and lower surfaces 32, 34.

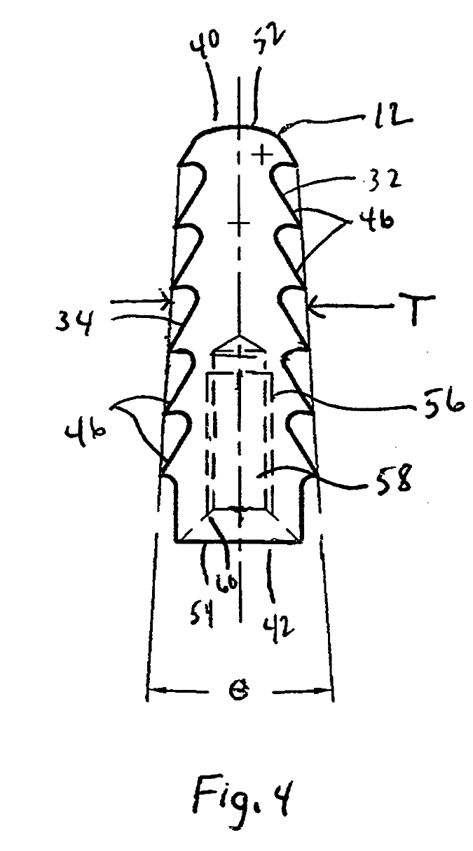

As shown in the side elevational view of FIG. 4, a convex surface 52 extends between the upper and lower surfaces 32, 34 at the posterior end 40 of the body 30. The convex surface 52 facilitates impaction of the broach 12 into the disc space between two vertebral bodies. A substantially planar surface 54 extends between upper and lower surfaces 32, 34 at the anterior end of the body 30. The planar surface 54 has a second fastening element 56 that is adapted to cooperate with the fastening element 24 of the inserter 14, to removably couple the broach 12 and the inserter 14 together in a secure manner. By way of example but not limitation, the second fastening element 56 may comprise a threaded closed-ended bore 58 with a beveled opening 60 (also see FIG. 3A), if the fastening element 24 of the inserter 14 comprises a threaded stud 25 used in the example above.

Referring still to FIG. 4, each tooth 46 angles back toward the anterior end 42 of the body 30. The upper and lower surfaces 32, 34 taper posteriorly at an angle 0 from the posterior-most pair of opposing teeth 46 such that the thickness T of the body 30, as measured between edges of the opposing teeth 46, decreases slightly from the anterior-most pair of opposing teeth 46 to the posterior-most pair of opposing teeth 46. This taper angle 0 matches the broach 12 to the implant lordosis, and in one exemplary embodiment, may be about 7 degrees.

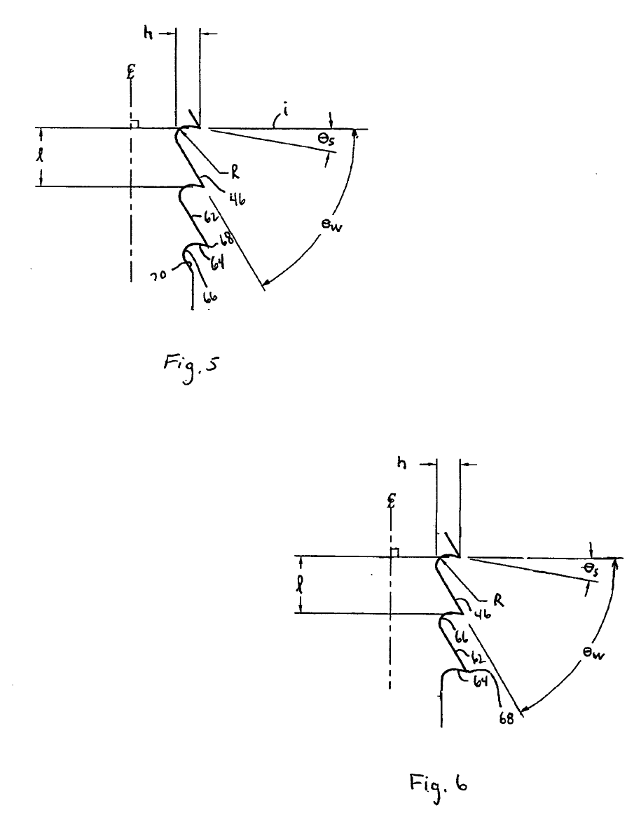

For purposes of illustration but not limitation, FIG. 5 shows an enlarged view of the two anterior-most teeth 46 (of the upper or lower body surface 32, 34) of an exemplary broach 12 in a 7 or 8 mm size (as measured between the posterior-most pair of opposing teeth). Each full tooth 46 typically includes a generally planar, anterior wedge surface 62, a generally planar posterior shovel surface 64, and a root surface 66. The intersection of the wedge and shovel surfaces 62, 64 define a sharp cutting edge 68. For purposes of illustration and not limitation, each tooth 46 may have a height h of about 0.064 inches, a length l of about 0.160 inches (except the two anterior-most opposing teeth 46 which each have a length l that is substantially less than 0.160 inches as can be seen in FIG. 4), and the root surface 66 may have a radius R of about 0.030 inches. Additionally, the wedge surface 62 of the tooth 46 is typically angled at an angle Ow of about 60°, as measured from an imaginary line i extending perpendicular to axial center line CL in the drawing, and the shovel surface 64 is typically angled at an angle Os of about 10°, as measured from the imaginary line i. Note that in this embodiment, a partial segment 70 of the wedge surface of another tooth extends from the root surface 66 of the anterior-most full tooth 46. This is necessary due to the small size of the broach and the need to have sufficient space for the threaded hole.

For purposes of illustration but not limitation, FIG. 6 shows an enlarged view of the two anterior-most teeth 46 of an exemplary broach 12 sized from 9 to 18 mm (as measured between the posterior-most pair of opposing teeth). The teeth 46 in this embodiment are substantially identical to the teeth 46 in the embodiment shown in FIG. 5, except there is no partial wedge surface of another tooth extending from the root surface 66 of the anterior-most tooth 46 (on larger sizes, there is sufficient space to have flats extend off of the root surface). Flats are preferred to provide clearance and for space to gather cut material.

Since vertebrae size and disc space vary from patient-to-patient (and since such sizes vary along the length of the spine of any given patient), several sizes of implants (not shown) are typically required. Additionally, the desired amount of distraction varies from patient to patient and from spine location to spine location. Therefore, several different sizes of broaches 12 for particular sizes of implants and/or amount of distraction are typically used together with one or more inserters 14 in a system according to the invention. There is a starter broach 12, as well as a broach 12 for each implant height and cross section. The broaches 12 may range in 1 mm increments from 7 to 18 mm (as measured between the posterior-most pair of opposing teeth). Each of the broaches 12 can be coupled to the same inserter 14, hence, only one inserter 14 is required in the system. However, one of ordinary skill in the art will of course appreciate that each broach 12 may be provided with its own inserter 14 in the system.

The use of the instrument system of the present invention will become apparent from the following description.

Preoperatively, the surgeon must decide which intervertebral levels to fuse. This may be done using a variety of diagnostic techniques, such as radiographs, MRI, discography, patient history, and physical examination.

For the L5/S1 level, the patient is positioned in the supine position, with a pad under the lumbar spine to maintain lordosis. For higher levels, the surgeon may choose either a supine position or a lateral decubitus position. In either case, a table should be used that accommodates both lateral and anterior-posterior radiographs.

A vascular or general surgeon usually provides the exposure while the spine surgeon assists and then performs the fusion. To access the L5/S1 level, the lumbar spine is exposed through a low transverse or paramedian incision while a retroperitoneal plane is developed. For higher levels, it may be more appropriate to use a midaxillary incision aligned over the level to be treated.

The implant/graft device is implanted in an anterior-posterior direction so that the retroperitoneal plane must be developed from the anterior direction.

Standard general and/or vascular surgical instruments are used to perform the exposure down to the level(s) of the fusion. They are also used to maintain the exposure via the appropriate retractors.

The exposure is completed when the anterior surface of the spine is exposed.

To identify the correct disc level(s) and that the approach is in the appropriate place, needle(s) are inserted into the intervertebral disc as markers, and the location(s) determined by means of a C-arm anterior-posterior radiograph. If the needle is not along the midline, it should be repositioned and an additional radiography taken to assure its proper location.

Iliac crest autograft is harvested from the iliac crest. This can be done using standard techniques. Depending on the size and height of the device used, approximately 0.5 to 1.5 cc3 of bone is required at each fusion level. The bone graft does not provide structural support but instead is used for its biological properties to stimulate bone formation.

An annulotomy is performed using a scalpel to make a window in the annulus. It should be centered about the midline. It should be as long as the space to be occupied by the implant.

Pituitary rangeurs and small currettes can be used to perform the discectomy.

Distraction and endplate preparation are performed using the instrument system of the present invention. Based on the size of the disc space, a broach 12 of the present invention is selected and coupled to the inserter of the invention. The broach 12 is impacted into the disc space by tapping on the proximal end surface 28 of the inserter handle 18 with a mallet. Intermittent lateral radiographic evaluation may be necessary to ensure the proper position in the anterior-posterior direction. Once the broach 12 is fully seated, it is removed from the disc space by moving the inserter in the medial-lateral direction perpendicular to the direction of insertion. A slap hammer is not used to remove the broach 12, in order to prevent fracturing the vertebral body or in damage to the anterior blood vessels. The teeth 46 on the broach 12 are back cutting, meaning that they only cut when the broach 12 is removed from the disc space. This prevents fragments from being pushed posteriorly towards the canal.

Broaching should be done in a step-wise fashion, working up from a shorter broach to a taller broach. The first broach should fit in the disc space and remove a minimal amount of bone. As soon as the endplate has been machined flat and bleeding bone exposed, endplate preparation has been completed.

One implant device is used at a single level. The proper implant size corresponds to the final broach used to prepare the disc space.

Placement of the implant device should be along the midline.

The hole of the appropriately sized implant is stuffed with the already harvested autologous bone. The implant is then coupled to an implant inserter. The inserter allows placement of the device into the disc space. A tamp is then used to fully seat the device. Moderate tapping on the tamp is required. If excessive force is needed, the implant is removed and disc space checked for obstructing bone or a narrow posterior opening. If excessive force is necessary, bone removal or a change in implant size may be required. The implant device should be slightly posterior of the anterior aspect of the vertebral bodies in its final position.

While the foregoing invention has been described with reference to the above embodiments, various modifications and changes can be made without departing from the spirit of the invention. Accordingly, all such modifications and changes are considered to be within the scope of the appended claims.

What is claimed is:

1. An instrument for distracting a disc space between adjacent vertebrae and simultaneously preparing endplates of the vertebrae, the instrument comprising:
a body having opposing upper and lower surfaces separated by curved side surfaces which extend between a posterior end of the body and an anterior end of the body;
a first plurality of teeth extending across the upper surface of the body, the first plurality of teeth all disposed in a first plane, the first plurality of teeth angling back toward the anterior end of the body, each of the teeth including an undercut;
a second plurality of teeth extending across the lower surface of the body, the second plurality of teeth all disposed in a second plane, the second plurality of teeth angling back toward the anterior end of the body, each of the teeth including an undercut; and
a body thickness measured between the first and second planes that continuously decreases from the anterior end to the posterior end.

2. The instrument according to claim 1, further comprising an inserter removably coupled to the body.

3. The instrument according to claim 1, where the first and second plurality of teeth further include posterior wedge surfaces and the undercuts comprise anterior shovel surfaces.

4. The instrument according to claim 3, wherein the first and second plurality of teeth further include arcuate root surfaces.

5. The instrument according to claim 3, wherein the wedge surfaces and the shovel surfaces intersect to define cutting edges.

6. The instrument according to claim 3, wherein the wedge surfaces are angled back at an angle of about 60°, as measured from an imaginary line extending perpendicular to an axis of the body, and the shovel surfaces are angled back at an angle of about 10°, as measured from the imaginary line.

7. The instrument according to claim 1, wherein the continuously decreasing body thickness defines a taper angle of about 7 degrees.

8. The instrument according to claim 1, wherein the body defines a broach.

9. A system for distracting a disc space between adjacent vertebrae and simultaneously preparing endplates of the vertebrae, the system comprising:
at least two differently dimensioned instruments, each of the instruments including:
a body having opposing upper and lower surfaces separated by curved side surfaces which extend between a posterior end of the body and an anterior end of the body;
a first plurality of teeth extending across the upper surface of the body, the first plurality of teeth all disposed in a first plane, the first plurality of teeth angling back toward the anterior end of the body, each of the teeth including an undercut;
a second plurality of teeth extending across the lower surface of the body, the second plurality of teeth all disposed in a second plate, the second plurality of teeth angling back toward the anterior end of the body, each of the teeth including an undercut; and
a body thickness measured between the first and second planes that continuously decreases from the anterior end to the posterior end.

10. The system according to claim 9, wherein the body of each instrument having an incrementally different average body thickness.

11. The system according to claim 9, further comprising an inserter removably coupled to the body of each instrument.

12. The instrument according to claim 9, where the first and second plurality of teeth further include posterior wedge surfaces and the undercuts comprise anterior shovel surfaces.

13. The system according to claim 12, wherein the first and second plurality of teeth further include arcuate root surfaces.

14. The system according to claim 12, wherein the wedge surfaces and the shovel surfaces intersect to define cutting edges.

15. The system according to claim 12, wherein the wedge surfaces are angled back at an angle of about 60°, as measured from an imaginary line extending perpendicular to an axis of the body, and the shovel surfaces are angled back at an angle of about 10°, as measured from the imaginary line.

16. The system according to claim 9, wherein the continuously decreasing body thickness defines a taper angle of about 7 degrees.

17. The system according to claim 9, wherein the body defines a broach.

18. An instrument for distracting a disc space between adjacent vertebrae and simultaneously preparing endplates of the vertebrae, the instrument comprising:
a body having opposing upper and lower surfaces separated by curved side surfaces which extend between a posterior end of the body and an anterior end of the body;
a first plurality of ratcheting teeth extending across the upper surface of the body, the first plurality of teeth all disposed in a first plane;
a second plurality of ratcheting teeth extending across the lower surface of the body, the second plurality of teeth all disposed in a second plane; and
a body thickness measured between the first and second planes that continuously decreases from the anterior end to the posterior end;
wherein the first and second plurality of ratcheting teeth are angled back toward the anterior end of the body to define a plurality of undercut surfaces.

19. The instrument according to claim 18, further comprising an inserter removably coupled to the body.

20. The instrument according to claim 18, where the first and second plurality of teeth further include posterior wedge surfaces and the undercuts comprise anterior shovel surfaces.

21. The instrument according to claim 20, wherein the first and second plurality of teeth further include arcuate root surfaces.

22. The instrument according to claim 20, wherein the wedge surfaces and the shovel surfaces intersect to define cutting edges.

23. The instrument according to claim 20, wherein the wedge surfaces are angled back toward the anterior end of the body at an angle of about 60°, as measured from an imaginary line extending perpendicular to an axis of the body, and the shovel surfaces are angled back toward the anterior end of the body at an angle of about 10°, as measured from the imaginary line.

24. The instrument according to claim 18, wherein the continuously decreasing body thickness defines a taper angle of about 7 degrees.

25. The instrument according to claim 18, wherein the body defines a broach.

26. A system for distracting a disc space between adjacent vertebrae and simultaneously preparing endplates of the vertebrae, the system comprising:
at least two differently dimensioned instruments, each of the instruments including:
a body having opposing upper and lower surfaces separated by curved side surfaces which extend between a posterior end of the body and an anterior end of the body;
a first plurality of ratcheting teeth extending across the upper surface of the body, the first plurality of teeth all disposed in a first plane;
a second plurality of ratcheting teeth extending across the lower surface of the body, the second plurality of teeth all disposed in a second plane; and
a body thickness measured between the first and second planes that continuously decreases from the anterior end to the posterior end;
wherein the first and second plurality of ratcheting teeth are angled back toward the anterior end of the body to provide a plurality of undercut surfaces.

27. The system according to claim 26, wherein the body of each instrument having an incrementally different average body thickness.

28. The system according to claim 26, further comprising an inserter removably coupled to the body.

29. The instrument according to claim 26, where the first and second plurality of teeth further include posterior wedge surfaces and the undercuts comprise anterior shovel surfaces.

30. The system according to claim 29, wherein the first and second plurality of teeth further include arcuate root surfaces.

31. The system according to claim 29, wherein the wedge surfaces and the shovel surfaces intersect to define cutting edges.

32. The system according to claim 29, wherein the wedge surfaces are angled back at an angle of about 60°, as measured from an imaginary line extending perpendicular to an axis of the body, and the shovel surfaces are angled back at an angle of about 10°, as measured from the imaginary line.

33. The system according to claim 26, wherein the continuously decreasing body thickness defines a taper angle of about 7 degrees.

34. The system according to claim 26, wherein the body defines a broach.

35. An instrument for distracting a disc space between adjacent vertebrae and simultaneously preparing endplates of the vertebrae, the system comprising:
a body having opposing upper and lower surfaces separated by curved side surfaces which extend between a posterior end of the body and an anterior end of the body;
a first plurality of teeth defined by the upper surface of the body, the first plurality of teeth all disposed in a first plane;
a second plurality of teeth defined by the lower surface of the body, the second plurality of teeth all disposed in a second plane; and
the body having a thickness measured between the first and second planes, the thickness continuously decreasing from an anterior-most pair of the first and second plurality of teeth to a posterior-most pair of the first and second plurality of teeth;
wherein the first and second plurality of teeth are angled back toward the anterior end of the body, thus creating an undercut surface associated with each of the teeth.

36. An instrument for distracting a disc space between adjacent vertebrae and simultaneously preparing endplates of the vertebrae, the system comprising:
at least two differently dimensioned instruments, each of the instruments including:
a body having opposing upper and lower surfaces separated by curved side surfaces which extend between a posterior end of the body and an anterior end of the body;
a first plurality of teeth defined by the upper surface of the body, the first plurality of teeth all disposed in a first plane;
a second plurality of teeth defined by the lower surface of the body, the second plurality of teeth all disposed in a second plane; and
the body having a thickness measured between the first and second planes, the thickness continuously decreasing from an anterior-most pair of the first and second plurality of teeth to a posterior-most pair of the first and second plurality of teeth;
wherein the first and second plurality of teeth are angled back toward the anterior end of the body, thus creating an undercut surface associated with each of the teeth.
